(19) United States
(12) Patent Application Publication
(10) Pub. No.: US 2022/0062575 A1
(43) Pub. Date: Mar. 3, 2022

(54) OROPHRANGEAL GLOVE FOR USE WITH RIGID AND FLEXIBLE BRONCHSCOPES, AND METHODS

(71) Applicant: Ismael A. Matus, Landenberg, PA (US)

(72) Inventor: Ismael A. Matus, Landenberg, PA (US)

(21) Appl. No.: 17/511,155

(22) Filed: Oct. 26, 2021

Related U.S. Application Data
(63) Continuation-in-part of application No. 16/045,396, filed on Jul. 25, 2018, now Pat. No. 11,224,337.

(51) Int. Cl. A61M 16/04; A61B 1/267

(52) U.S. Cl. CPC A61M 16/0497; A61B 1/2676; A61M 16/0402

(57) ABSTRACT

An oropharyngeal glove (OPG) is provided for use in a rigid or flexible bronchoscopy procedure and for anesthesia recovery. Portions of the OPG conform to portions of the patient's mouth and throat. During a bronchoscopy procedure, the bronchoscope tube passes through an opening in a proximal end of the OPG, through the OPG and through an opening in a distal end of the OPG into the patient's trachea. A protective lining of the OPG protects the patient's mouth, throat and vocal cords from being damaged by the bronchoscope tube. A tubular extension disposed on the proximal end of the OPG provides the opening through which the bronchoscope tube first passes. Post procedure, the tubular extension can act as an airway device that connects to a ventilator machine to deliver air to the patient, thereby obviating the need to install a separate airway device to ventilate the patient during anesthesia recovery.

### CROSS-REFERENCE TO RELATED APPLICATIONS

The present application is a continuation-in-part (CIP) application of U.S. application Ser. No. 16/045,396, filed on Jul. 25, 2018, entitled “AN OROPHRANGEAL GLOVE FOR USE WITH A RIGID BRONCHSCOPE AND A METHOD,” which has been allowed, and which is incorporated by reference herein.

### TECHNICAL FIELD

Bronchoscopy is an endoscopic process that involves visualizing the inside of the airways for therapeutic and diagnostic purposes. An instrument known as a bronchoscope is inserted through the patient's mouth into the airways to allow the physician to examine the airways for abnormalities such as bleeding, tumors or inflammation, for example.

### BACKGROUND

Bronchoscopy is an endoscopic process that involves visualizing the inside of the airways for therapeutic and diagnostic purposes. An instrument known as a bronchoscope is inserted through the patient's mouth into the airways to allow the physician to examine the airways for abnormalities such as bleeding, tumors or inflammation, for example.

Generally, there are two types of bronchoscopes: flexible bronchoscopes and rigid bronchoscopes. Flexible bronchoscopes have a fiber optic system that transmits an image from the end of an optical fiber that is inside of the patient to an eyepiece or camera at the opposite end of the optical fiber. Rigid bronchoscopes have a larger lumen than that of the flexible bronchoscope and are typically made of a hard metallic material.

There are problems that can occur when performing rigid bronchoscopy. Because of the rigid nature of the instrument, it can sometimes cause abrasions or lacerations to the patient's mouth, throat or vocal cords and can damage the patient's teeth.

Also, as rigid bronchoscopic procedures are performed under general anesthesia and are considered “open circuit” procedures, varying degrees of air leaks exist not only through the barrel of the rigid bronchoscope to the atmosphere, but also from the patient's airway around the rigid bronchoscope which can challenge ventilation of the patient.

In addition to the problems discussed above, both rigid and flexible bronchoscopy procedures require use of a separate airway device post procedure for ventilating the patient while the patient recovers from anesthesia. During a rigid bronchoscopy procedure, air is supplied to the patient's airway through the tube of the rigid bronchoscope. Once the procedure is completed, the rigid bronchoscope is removed and an airway device, such as an igel® supraglottic airway device with mask, an endotracheal tube or other type of airway device, that is connected to a ventilator is attached to the patient to resume ventilation of the patient. During a flexible bronchoscopy procedure, such a mask or endotracheal tube is used and the tube of the flexible bronchoscope is fed into the patient through an uncapped port of the airway device. When the bronchoscopy procedure is complete, the flexible bronchoscope tube is removed from the patient and from the airway device and the uncapped port of the airway device is capped. Air delivered by the airway device continues to ventilate the patient until the patient is recovered.

There is significant cost associated with purchasing the separate airway device and having a healthcare professional apply it to the patient. A need exists for a medical device that can be used when performing bronchoscopy to protect the patient's mouth, throat, vocal cords and teeth, that reduces or eliminates the possibility of air leakage during the procedure and that obviates the need for a separate airway device to perform post-procedure anesthesia recovery.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a side transparency view of a patient with an oropharyngeal glove installed in the patient's mouth and throat.

FIG. 2 is a side cross-sectional view of the oropharyngeal glove shown in FIG. 1 in its uninstalled state and having a generally tubular portion of a rigid bronchoscope inserted through a first opening formed in a proximal end of the oropharyngeal glove.

FIG. 3 is a side cross-sectional view of the oropharyngeal glove shown in FIG. 1 in its uninstalled state as shown in FIG. 2 and having the generally tubular portion of a rigid bronchoscope shown in FIG. 2 passing through a slit opening formed in a distal end of the oropharyngeal glove.

FIG. 4 is a side transparency view of a patient with the oropharyngeal glove installed in the patient's mouth and throat with a generally tubular portion of a rigid bronchoscope passing through the oropharyngeal glove from the proximal end thereof through a second opening formed in the distal end thereof into the patient's trachea.

FIG. 5 is a side cross-sectional view of an inflatable oropharyngeal glove in accordance with another embodiment in its uninstalled state, and with a generally tubular portion of a rigid bronchoscope through an opening formed in a proximal end of the glove.

FIG. 6 illustrates a flow diagram of the method, in accordance with a representative embodiment, for performing a rigid bronchoscopy procedure using the oropharyngeal glove shown in FIGS. 1-4.

FIG. 7 illustrates a flow diagram of the method, in accordance with a representative embodiment, for performing a rigid bronchoscopy procedure using the oropharyngeal glove shown in FIG. 5.

FIG. 8 is a side cross-sectional view of the oropharyngeal glove shown in FIG. 2 in accordance with a representative embodiment in which the glove has been modified to include a tubular extension that is adapted to be coupled to an airway device to enable the glove to be used post procedure as part of the airway circuit to continue ventilating the patient until the patient has recovered from anesthesia.

FIG. 9 is a front-end perspective view of the tubular extension of the oropharyngeal glove shown in FIG. 8.

FIG. 10 is a side transparency view of a patient with the oropharyngeal glove shown in FIG. 8 in accordance with a representative embodiment installed in the patient's mouth and throat.

FIG. 11 is a side cross-sectional view of the second membrane portion of the oropharyngeal glove shown in FIG. 8 in accordance with a representative embodiment in which the distal end of the glove has been modified to be removably attached to a removably-attachable distal end port to enable the glove to be used for rigid bronchoscopy or flexible bronchoscopy.

FIG. 12 is a side plan view of a removably-attachable distal end port in accordance with a representative embodiment adapted to be removably attached to the distal end of the glove shown in FIG. 11 to enable the glove to be used for rigid bronchoscopy.

FIG. 13 is a side plan view of a removably-attachable distal end port in accordance with a representative embodiment adapted to be removably attached to the distal end of the glove shown in FIG. 11 to enable the glove to be used for flexible bronchoscopy.

FIG. 14 is a side cross-sectional view of the second membrane portion of the oropharyngeal glove shown in FIG. 11 in accordance with a representative embodiment in which the distal end of the glove is coupled with the removably-attachable distal end port shown in FIG. 12 to enable the glove to be used for rigid bronchoscopy.

FIG. 15 is a side plan view of the second membrane portion of the oropharyngeal glove shown in FIG. 11 in accordance with a representative embodiment in which the distal end of the glove is coupled with the removably-attachable distal end port shown in FIG. 13 to enable the glove to be used for flexible bronchoscopy.

FIG. 16 is a bottom end view of a cross-section of the removably-attachable distal end port shown in FIG. 15 taken along line A-A′ of FIG. 15 in accordance with a representative embodiment.

FIGS. 17A and 17B show bottom end and side plan views, respectively, of the distal end of the glove shown in FIG. 8 in which the distal end of the glove has been modified to slidably engage a removably-attachable distal end port to enable the glove to be used for flexible bronchoscopy.

FIGS. 18A and 18B show bottom end and side plan views, respectively, of a removably-attachable distal end port in accordance with another representative embodiment adapted to slidably engage the distal end of the glove shown in FIGS. 17A and 17B to enable the glove to be used for flexible bronchoscopy.

### DETAILED DESCRIPTION

The present disclosure is directed to various representative embodiments of an oropharyngeal glove (OPG) for use with rigid and flexible bronchoscopes, as well as representative embodiment of methods of using the glove to perform rigid and flexible bronchoscopy procedures. Portions of the OPG conform to portions of the patient's mouth and throat. During a bronthoscopy procedure, the bronchoscope tube passes through an opening in a proximal end of the OPG, through the OPG and through an opening in a distal end of the OPG into the patient's trachea. A protective lining of the OPG protects the patient's mouth, throat and vocal cords from being damaged by the bronchoscope tube. A tubular extension disposed on the proximal end of the OPG provides the opening through which the bronchoscope tube first passes into the OPG. Post procedure, the tubular extension can act as an airway device that connects to a ventilator machine via a breathing circuit to deliver air to the patient, thereby obviating the need to install a separate airway device to ventilate the patient during anesthesia recovery.

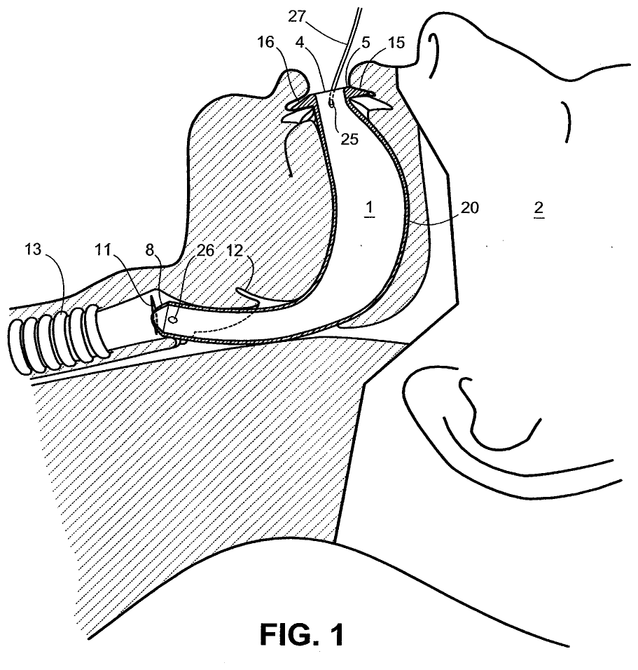

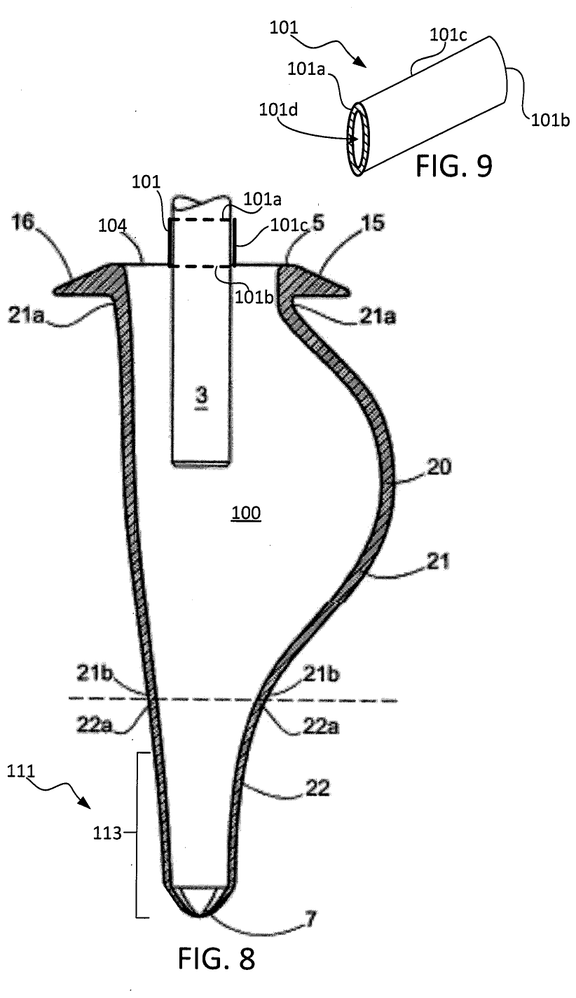

As indicated above, the present application is a CIP application of U.S. application Ser. No. 16/045,396 (hereinafter referred to as “the parent case”). The present CIP case discloses representative embodiments in which the glove is adapted to obviate the need for a separate mask-type airway device to perform post-procedure anesthesia recovery, as well as representative embodiments in which a distal end of the OPG is modified to couple with a removably-attachable distal end port to allow different distal end ports to be attached to the distal end of the OPG to make it suitable for either rigid bronchoscopy or flexible bronchoscopy, depending on the type of bronchoscopy procedure that is to be performed. FIGS. 1-7 from the parent case and the corresponding text are included herein for completeness. The new representative embodiments of the present CIP disclosure are shown in FIGS. 8-18B and discussed below in detail.

In the following detailed description, for purposes of explanation and not limitation, exemplary, or representative, embodiments disclosing specific details are set forth in order to provide a thorough understanding of the inventive principles and concepts. However, it will be apparent to one of ordinary skill in the art having the benefit of the present disclosure that other embodiments according to the present teachings that are not explicitly described or shown herein are within the scope of the appended claims. Moreover, descriptions of well-known apparatuses and methods may be omitted so as not to obscure the description of the exemplary embodiments. Such methods and apparatuses are clearly within the scope of the present teachings, as will be understood by those of skill in the art. It should also be understood that the word “example,” as used herein, is intended to be non-exclusionary and non-limiting in nature.

The terminology used herein is for purposes of describing particular embodiments only and is not intended to be limiting. Any specifically-defined terms are in addition to the technical, scientific, or ordinary meanings of the defined terms as commonly understood and accepted in the relevant context.

The terms “a,” “an” and “the” include both singular and plural referents, unless the context clearly dictates otherwise. Thus, for example, “a device” includes one device and plural devices. The terms “substantial” or “substantially” mean to within acceptable limits or degrees acceptable to those of skill in the art. For example, the term “substantially parallel to” means that a structure or device may not be made perfectly parallel to some other structure or device due to tolerances or imperfections in the process by which the structures or devices are made. The term “approximately” means to within an acceptable limit or amount to one of ordinary skill in the art.

Relative terms, such as “over,” “above,” “below,” “top,” “bottom,” “front,” “back,” “upper” and “lower” may be used to describe the various elements' relationships to one another, as illustrated in the accompanying drawings. These relative terms are intended to encompass different orientations of the device and/or elements in addition to the orientation depicted in the drawings. For example, if the device were inverted with respect to the view in the drawings, an element described as “above” another element, for example, would now be below that element.

The term “bronchoscope,” as that term is used herein, can mean a rigid bronchoscope or a flexible bronchoscope unless specifically referred to herein as a “rigid bronchoscope” or a “flexible bronchoscope.” A “rigid bronchoscope,” as that term is used herein, means a bronchoscope having a rigid tube, as is known in the art of bronchoscopy. A “flexible bronchoscope,” as that term is used herein, means a bronchoscope having a flexible tube, as is known in the art of bronchoscopy.

When the oropharyngeal glove (referred to herein as “the OPG”) is in its installed state, it conforms to the patient's mouth and throat. in preferred embodiments, the OPG includes upper and lower teeth guards that are in contact with the patient's upper and lower front teeth, respectively, when the OPG is in the installed state. The OPG has a first opening disposed in its proximal end that allows the bronchoscope tube to enter the OPG. The OPG has a second opening formed in its distal end through which the bronchoscope tube passes to enter the patient's trachea. The portions of the OPG that conform to the patient's mouth and throat comprise a protective lining that protects the mouth, throat and vocal cords from being damaged by the rigid bronchoscope. The upper and lower teeth guards protect the patient's upper and lower front teeth, respectively, from being damaged by the bronchoscope tube during the bronchoscopy procedure and during installation and removal of the bronchoscope. It should he noted that the teeth guards are preferred, but not required, in eases where the OPG is being used with a rigid bronchoscope having a rigid tube to protect the teeth, but the teeth guards may not he needed and therefore may not be part of the OPG when the OPG is being used with a flexible bronchoscope having a flexible tube.

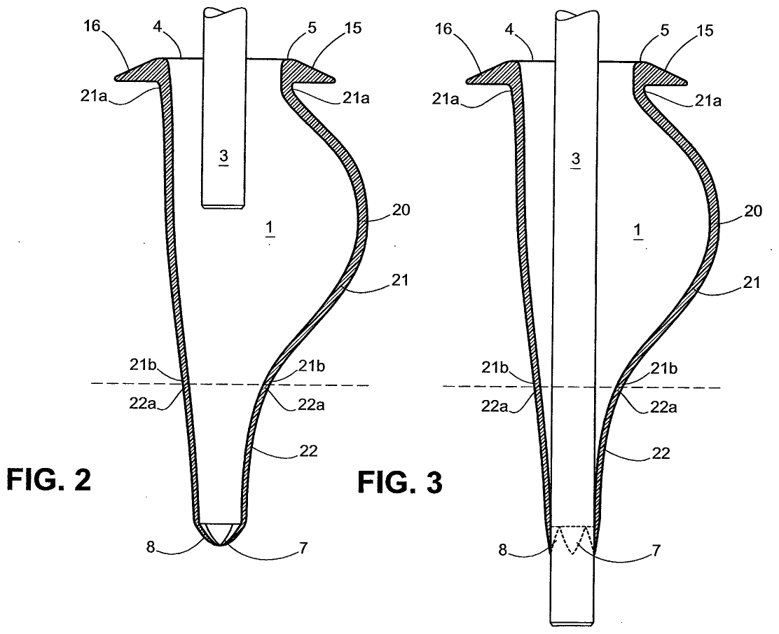

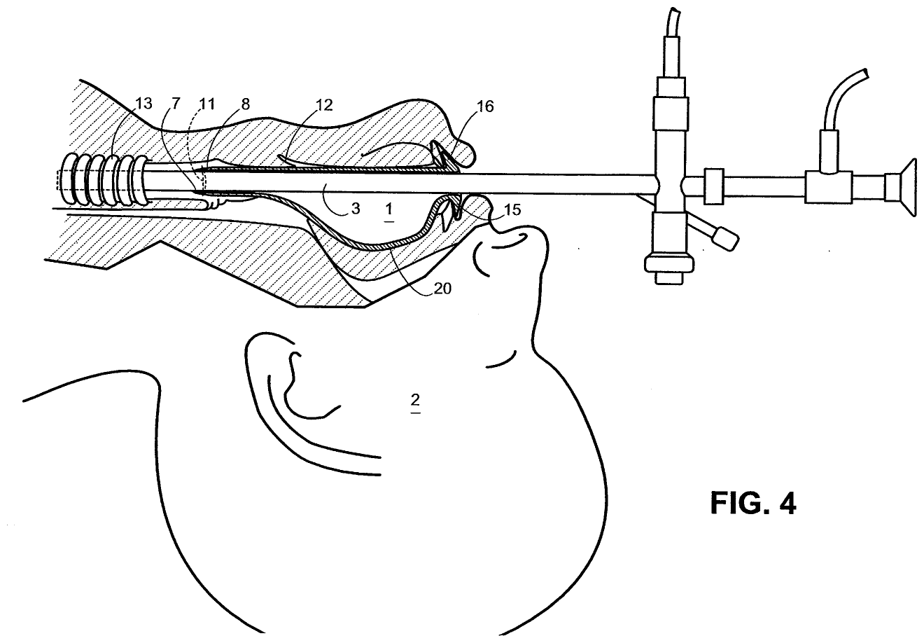

FIG. 1 is a side transparency view of a patient 2 with the OPG 1 in accordance with a representative embodiment installed in the patient's mouth and throat. The OPG 1 in accordance with this representative embodiment is particularly well suited for rigid bronchoscopy. FIG. 2 is a side cross-sectional view of the OPG 1 shown in FIG. 1 in its uninstalled state and having a generally tubular portion 3 of a rigid bronchoscope inserted through a first opening 4 formed in a proximal end 5 of the OPG 1. FIG. 3 is a side cross-sectional view of the OPG shown in FIG. 1 in its uninstalled state as shown in FIG. 2 with the generally tubular portion 3 of a rigid bronchoscope passing through a second opening 7 formed in a distal end 8 of the OPG 1. FIG. 4 is a side transparency view of the patient 2 with the OPG 1 installed in the patient's mouth and throat with the generally tubular portion 3 of the rigid bronchoscope passing through the OPG 1 from the proximal end 5 thereof through the second opening 7 formed in the distal end 8 thereof into the patient's trachea 13.

In the installed state of the OPG 1, the distal end 8 preferably is positioned just above, or flush with, the vocal cords 11 and below the epiglottis 12. In other words, in the installed state of the OPG 1, the distal end 8 preferably is positioned flush with the vocal cords 11 or in between the vocal cords 11 and the epiglottis 12. In this position, the second opening 7 formed in the distal end 8 is aligned with the trachea 13 and the OPG 1 blocks the entryway of the esophagus.

The OPG 1 preferably has upper and lower teeth guards 15 and 16, respectively, that are in contact with the patient's upper and lower front teeth, respectively, when the OPG 1 is in the installed state shown in FIG. 1. The OPG 1 has a flexible body 20 that extends from the upper and lower teeth guards 15 and 16, respectively, to the distal end 8 of the OPG. The flexible body 20 is configured to conform to a patient's mouth and throat when the OPG 1 is installed in the patient's mouth and throat such that outer walls of the flexible body 20 are in contact with the patient's tongue, with the inner walls of the patient's cheeks, with the roof of the patient's mouth, and with the patient's throat. Preferably, the outer surface of the flexible body 20 forms an airtight, or nearly airtight, seal with these anatomical features of the patient's mouth and throat.

In accordance with a representative embodiment, the flexible body 20 of the OPG 1 comprises a first membrane portion 21 (FIGS. 2 and 3) and a second membrane portion 22 (FIGS. 2 and 3). The first membrane portion 21 has a first end 21a that meets the upper and lower teeth guards 15 and 16, respectively. The teeth guards 15 and 16 and the first end 21a of the first membrane portion 21 define the first opening 4. The first membrane portion 21 has a second end 21b that meets a first end 22a of the second membrane portion 22. The second membrane portion 22 has a second end 22b that meets the distal end 8 of the OPG 1. The term “meets,” as that term is used herein, can have multiple meanings. The term “meets” can mean that there is a physical joining of different elements or features of different materials, a contiguous transition of one element or feature into another element or feature made of the same material based on a preselected or arbitrary boundary, a joining of different elements or features via an attachment mechanism, such as an adhesive material, for example, etc.

When the OPG 1 is installed in a patient's mouth and throat, as shown in FIG. 1, outer walls of the first membrane portion 21 are in contact with the patient's tongue, with the inner walls of the patient's cheeks and with the roof of the patient's mouth, and outer walls of the second membrane portion 22 are in contact with the patient's throat. As indicated above, this contact preferably forms an airtight, or nearly airtight, seal.

In accordance with a representative embodiment, in the installed and uninstalled states, the first membrane portion 21 has a width, or circumference, that is greater than a width, or circumference, of the second membrane portion 22. The reason for this is that the inside of the mouth is wider than the inside of the throat. The first and second membrane portions 21 and 22, respectively, are flexible to allow the OPG 1 to be bent, folded or compressed while it is being installed in the patient's mouth or throat. The person performing the bronchoscopy procedure may install the OPG 1 by hand or by using a tool (not shown) to temporarily deform the OPG 1 to enable it to be inserted into the proper position shown in FIG. 1. The OPG 1 is made of a material that has some memory so that the OPG 1 attempts to return to the state it was in before being temporarily deformed. In the installed state of the OPG 1 shown in FIG. 1, the second membrane portion 22 blocks the entryway of the esophagus of the patient and the outer surfaces of the first and second membrane portions 21 and 22, respectively, create an airtight, or nearly airtight, seal with the surfaces of the mouth and throat, respectively.

In accordance with a representative embodiment, the OPG 1 includes at least a first oxygen port 25 (FIG. 1) disposed at the proximal end 5 of the OPG 1 and at least a second oxygen port 26 (FIG. 1) disposed at the distal end 8 of the OPG 1. The first and second oxygen ports 25 and 26, respectively, are interconnected via a conduit disposed in the walls of the flexible body 20. Oxygen may be delivered via a tube 27 to the first oxygen port 25. Oxygen delivered to the first oxygen port 25 will flow through the conduit to the second oxygen port 26. During the bronchoscopy procedure, oxygen received in the oxygen port 26 from the first oxygen port 25 will flow out of the second oxygen port 26 near the entryway of the trachea 13, thereby providing additional oxygen to the patient's lungs.

In accordance with a representative embodiment, the second opening 7 (FIGS. 2 and 3) is a slit opening, as depicted in FIGS. 2-4. The slit opening opens just wide enough to allow the tubular portion 3 of the rigid bronchoscope to pass through it and forms a tight friction fit about the tubular portion 3. The friction fit is tight enough to substantially seal the slit opening to prevent air from passing through the opening.

In accordance with an embodiment, the OPG 1 is an integrally-formed part, although the OPG 1 could comprise separate parts that are joined together by some suitable process and mechanism. The OPG 1 may be made of any suitable material, such as a medical-grade plastic or a synthetic rubber, for example. In any case, the upper and lower teeth guards 15 and 16, respectively, will typically be the hardest and least flexible elements of the OPG 1, which can be accomplished by making those elements thicker and/or denser than any of the other elements. The first and second membrane portions 21 and 22, respectively, could be made of the same material (e.g., medical-grade plastic or rubber), but with the second membrane portion 22 being more flexible than the first membrane portion 21. This can be accomplished in a number of ways, such as, for example, by making the walls of the second membrane portion 22 thinner than the walls of the first membrane portion 21. This is demonstrated in FIGS. 2 and 3, which show that the walls of the flexible body 20 in the first membrane portion 21 are thicker than the walls of the flexible body 20 in the second membrane portion 22.

Persons of skill in the art will understand, in view of the considerations described herein, how to select a suitable material and manufacturing process to form the OPG 1 to have the desired characteristics of flexibility, conformity and strength. For example, a plastic molding process can be used to form the OPG 1 as an integrally-formed, or unitary, part. As another example, an epoxy replication process may be used to form the OPG 1. As indicated above, the OPG 1 may be made of different materials. For example, the OPG 1 may be made of plastic and rubber. Thus, the inventive principles and concepts are not limited with respect to the material(s) that is used to make the OPG 1 or with respect to the process that is used to make the OPG 1, as will be understood by those of skill in the art in view of the description provided herein.

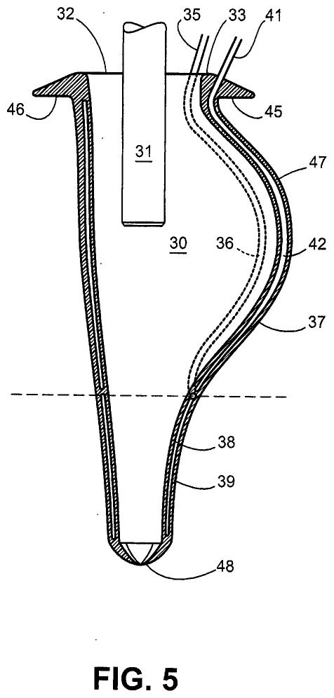

FIG. 5 is a side cross-sectional view of an inflatable OPG 30 in accordance with another representative embodiment in its uninstalled state with a generally tubular portion 31 of a rigid bronchoscope inserted through an opening 32 formed in a proximal end 33 of the OPG 30. In FIG. 5, although the OPG 30 is shown in its uninstalled state, it is shown inflated to demonstrate its shape when it is in the inflated state. However, the OPG 30 will be installed in the patient's throat and mouth when it is in a deflated state. In the deflated state, the OPG 30 can be compressed into a suitable compressed shape for insertion into the patient's mouth and throat.

Once the OPG 30 has been installed, air or another suitable gas supplied via a first tube 35 is carried via a first conduit 36 through the wall of the first membrane portion 37 of the OPG 30 to a first pocket 38 disposed in the wall of the second membrane portion 39. This causes the second membrane portion 39 to insufflate, which causes the circumference of the second membrane portion 39 to expand, i.e., to widen. Air or another suitable gas supplied via a second tube 41 is supplied to a second pocket 42 disposed in the wall of the first membrane portion 37. This causes the first membrane portion 37 to insufflate, which causes the circumference of the first membrane portion 37 to expand, i.e., to widen. Widening the first and second membrane portions 37 and 39, respectively, in this manner causes the first and second membrane portions 37 and 39, respectively, to conform the inner surfaces of the mouth and throat, respectively, to create an airtight, or nearly airtight, seal between these surfaces and the outer surfaces of the first and second membrane portions 37 and 39, respectively.

One of the benefits of the inflatable OPG 30 shown in FIG. 5 is that it can be installed or deployed without the need for a tool. Another benefit is that the OPG 30 will work with patients of different sizes because the amount to which the OPG 30 is inflated can be controlled to ensure that the OPG 30 is inflated to the correct size to fit the patient's mouth and throat. In other words, for smaller patients, the OPG 30 will be inflated to a lesser degree than for larger patients. As with the OPG 1 shown in FIGS. 1-4, the OPG 30 can be made of any suitable material (e.g., medical-grade plastic and/or synthetic rubber) and can be made by any suitable process (e.g., molding).

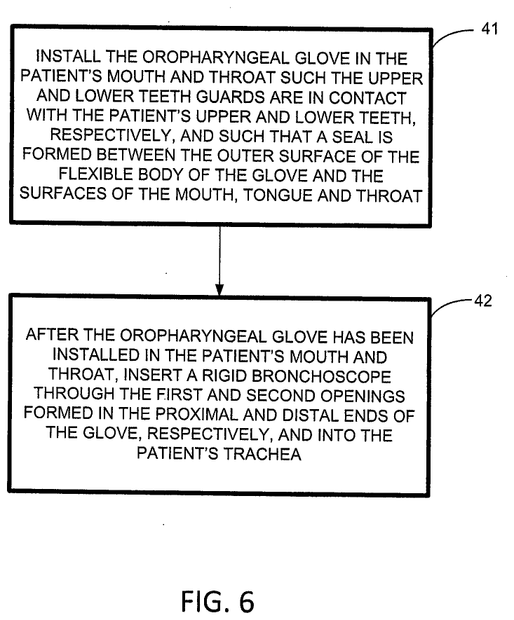

The method, in accordance with a representative embodiment, for performing a rigid bronchoscopy procedure using the OPG 1 will now be described with reference to FIG. 6. The OPG 1 is installed in the patient's mouth and throat such that the upper and lower teeth guards 15 and 16, respectively, are in contact with the upper and lower teeth, respectively, and such that an airtight, or nearly airtight, seal is formed between the outer surface of the flexible body 20 and the surfaces of the mouth, tongue and throat, as indicated by block 41. After the OPG 1 has been installed, a generally tubular portion of the rigid bronchoscope is inserted through the first and second openings 4 and 7, respectively, and into the patient's trachea 13, as indicated by block 42. The OPG 1 protects the mouth, teeth and throat of the patient from being damaged by the bronchoscope.

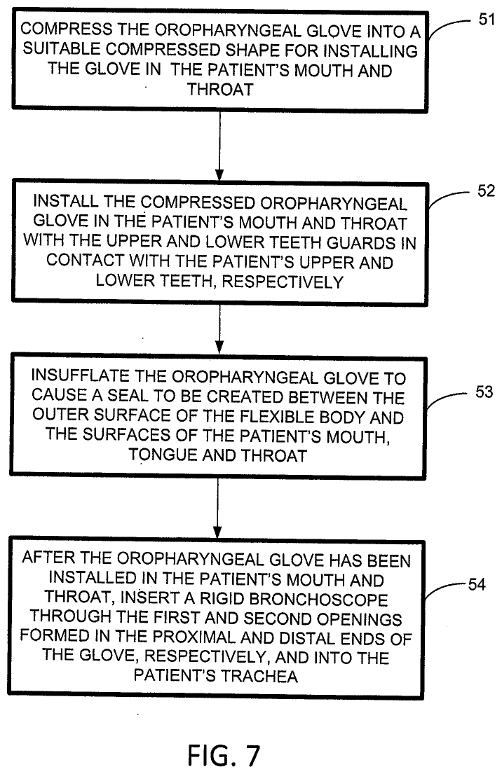

The method, in accordance with another representative embodiment, for performing a rigid bronchoscopy procedure using the OPG 30 will now be described with reference to FIG. 7. The OPG 30 is compressed into a suitable compressed shape and installed in the patient's mouth and throat, as indicated by block 51. The OPG 1 is installed in the patient's mouth and throat with the upper and lower teeth guards 45 and 46, respectively, in contact with the upper and lower teeth, respectively, as indicated by block 52. The OPG 30 is then insufflated to cause an airtight, or nearly airtight, seal to be formed between the outer surface of the flexible body 47 of the OPG 30 and the surfaces of the mouth, tongue and throat, as indicated by block 53. After the OPG 30 has been installed in this manner, a generally tubular portion of the rigid bronchoscope is inserted through the first and second openings 32 and 48, respectively, and into the patient's trachea 13, as indicated by block 54. The OPG 30 protects the mouth, teeth and throat of the patient from being damaged by the bronchoscope.

FIG. 8 is a side cross-sectional view of an OPG 100 in accordance with a representative embodiment that is similar in many regards to the OPG 1 shown in FIG. 2, except that the OPG 100 includes a tubular extension 101 that is adapted to be coupled with a breathing tube of a breathing circuit of a ventilator to enable the OPG 100 to be used post-procedure as part of the airway circuit to continue ventilating the patient until the patient has recovered from anesthesia. In FIG. 8, the generally tubular portion 3 of a rigid bronchoscope is shown inserted through the tubular extension 101. FIG. 9 is a front-end perspective view of the tubular extension 101. FIG. 10 is a side transparency view, similar to the view shown in FIG. 1, of a patient with the OPG 100 shown in FIGS. 8 and 9 installed in the patient's mouth and throat.

By performing this additional anesthesia recovery function, the OPG 100 obviates the need for a separate airway device for performing anesthesia recovery. As indicated above, currently, both rigid and flexible bronchoscopy procedures require use of a separate airway device post procedure for ventilating the patient while the patient recovers from anesthesia. Currently, during a rigid bronchoscopy procedure, air is supplied to the patient's airway through the rigid tube of the rigid bronchoscope. Once the bronchoscopy procedure is completed, the rigid bronchoscope is removed and an airway device, such as, for example, an igel® supraglottic airway device, an endotracheal tube device or other type of airway device, is coupled via a breathing circuit to a ventilator machine to resume ventilation of the patient. During a flexible bronchoscopy procedure, such a mask or endotracheal tube is used and the flexible tube of the flexible bronchoscope is fed into the patient through an uncapped port of the airway device. When the flexible bronchoscopy procedure is complete, the flexible bronchoscope tube is removed from the patient and from the airway device and the uncapped port of the airway device is capped. Air delivered by the airway device to the patient continues to ventilate the patient until the patient is recovered.

As indicated above, the OPG 100 obviates the need for the additional airway device. In accordance with this representative embodiment, the tubular extension 101 is sized to mate with a known breathing tube of a breathing circuit of a known ventilator machine. Sizing the tubular extension 101 to mate with a known breathing tube allows the breathing tube that is coupled on one end to the ventilator machine to simply be coupled on its opposite end to the proximal end of the tubular extension 101 to allow ventilation of the patient to continue until the patient has recovered from anesthesia.

In accordance with a representative embodiment, the tubular extension 101 is tubular, i.e., cylindrical in shape, has an outer diameter and an inner diameter, a proximal end 101a, a distal end 101b, and a tubular section 101c that extends from the proximal end 101a to the distal end 101b. The tubular section 101c has a hollow inner bore 101d defined by the inner diameter of the tubular extension 101. The outer diameter of the tubular extension 101 can be substantially equal to the inner diameter of the aforementioned breathing tube, or vice versa, to allow the extension 101 to easily mate with the end of the breathing tube in an airtight arrangement. The inner diameter of the extension 101 preferably is sufficiently large to accommodate the width or diameter of a rigid bronchoscope tube, at least in cases where the OPG 100 is intended to be used for rigid bronchoscopy and for cases where the OPG 100 is intended to be used for rigid and flexible bronchoscopy. Rigid bronchoscope tubes are larger in width or outer diameter than flexible bronchoscope tubes. For cases where the OPG 100 is intended to be used for flexible, but not rigid, bronchoscopy, the inner diameter of the extension 101 need only be large enough to accommodate a flexible bronchoscope tube. For purposes of describing the inventive principles and concepts of the present disclosure, it will be assumed that the OPG 100 is configured to be sufficiently versatile to be suitable for rigid and flexible bronchoscopy, and therefor that the extension 101 has an inner diameter that is sufficiently large to accommodate a rigid bronchoscope tube.

It should be noted that the inventive principles and concepts of the present disclosure are not limited to the tubular extension 101 having any particular dimensions. However, providing the tubular extension 101 with an outer diameter that is suitable for mating the extension 101 with a known breathing tube of a known ventilator machine breathing circuit reduces costs and complexity by eliminating the need to use an adapter to achieve an airtight interface between the breathing circuit tube and the proximal end 101a of the tubular extension 101. It should be noted, however, that it is within the scope of the inventive principles and concepts of the present disclosure to employ an adapter for such a purpose.

All of the features of the OPG 100 shown in FIGS. 8 and 9, other than the tubular extension 101, can be the same as those of the OPG 1 shown in FIGS. 1, 2 and 3 and described above. In the OPG 100 shown in FIGS. 8 and 9, the first opening 4 shown in FIGS. 2 and 3 no longer exists and the only opening in the proximal end 5 of the OPG 100 is the opening defined by the inner diameter of the tubular extension 101, which passes through and is secured to material 104 of the OPG 130. The inner and outer surfaces of the tubular extension 101 defined by the inner and outer diameters, respectively, of the tubular extension 101 are typically coaxial with one another and coaxial with a central axis of the tubular extension 101, although this is not necessarily the case in all embodiments, as there is no need for the inner and outer surfaces to be concentric. Also, while the size of the inner diameter of the tubular extension 101, which defines the hollow inner bore, is typically constant, the size of the inner diameter can vary along the length of the hollow inner bore in some embodiments. Likewise, the size of the outer diameter of the tubular extension 101 is typically constant along the length of the extension 101, but it can also vary along that length in some embodiments.

An additional benefit of using the OPG 100 for anesthesia recovery is that when the OPG 100 is in the installed state depicted in FIG. 10, one or regions of the OPG 100 in an area 113 along the distal end 111 of the OPG 100 seals off the upper aperture of the esophagus to prevent aspiration of gastric contents into the patient's airway while under anesthesia. By allowing the OPG 100 to remain in the installed state post-bronchoscopy during anesthesia recovery while air is being provided to the patient's airway via the tubular extension 101.

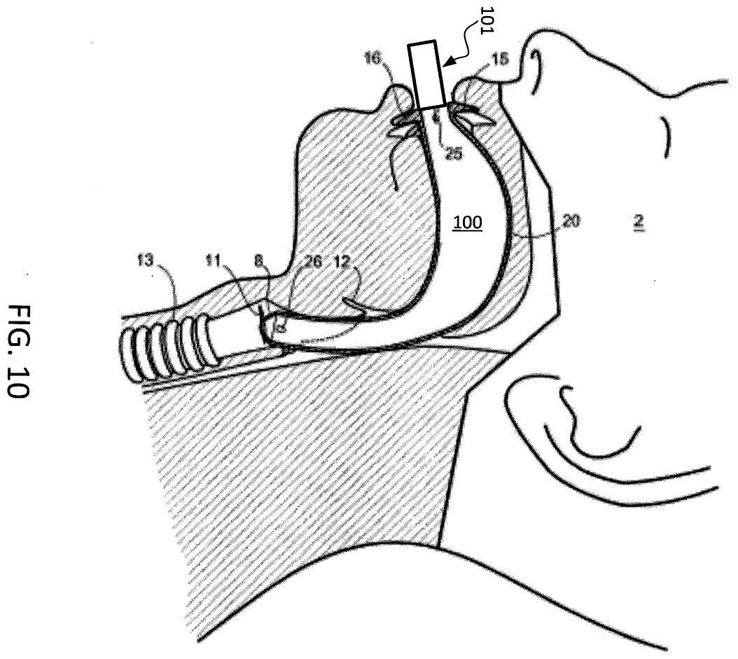

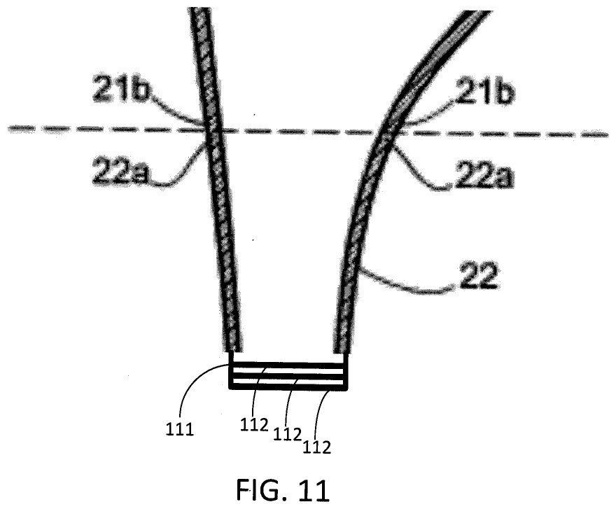

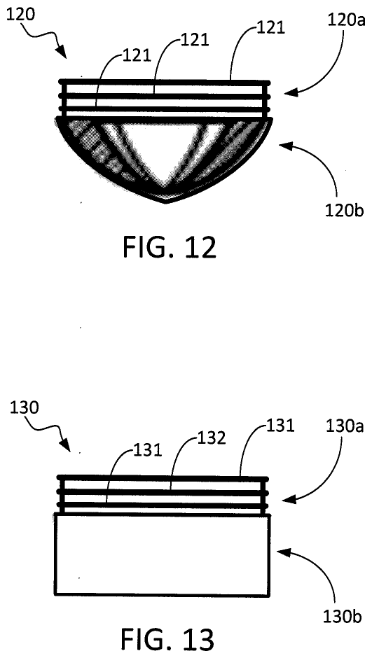

FIG. 11 is a side cross-sectional view of the second membrane portion 22 of the OPG 100 shown in FIG. 8 in accordance with a representative embodiment in which the distal end 111 of the OPG 100 has been modified to be removably attached to a removably-attachable distal end port to enable the OPG 100 to be used for rigid bronchoscopy or flexible bronchoscopy. FIG. 12 is a side plan view of a removably-attachable distal end port 120 in accordance with a representative embodiment adapted to be removably attached to the distal end 111 of the OPG 100 shown in FIG. 11 to enable the glove to be used for rigid bronchoscopy. FIG. 13 is a side plan view of a removably-attachable distal end port 130 in accordance with a representative embodiment adapted to be removably attached to the distal end 111 of the OPG 100 shown in FIG. 11 to enable the OPG 100 to be used for flexible bronchoscopy.

As shown in FIG. 11, the inner surface of the distal end 111 of the OPG 100 is threaded with threads 112, i.e., it is a female threaded surface. As shown in FIGS. 12 and 13, upper portions 121a and 131a of the outer surfaces of the removably-attachable distal end ports 120 and 130, respectively, are threaded with threads 121 and 131, respectively, i.e., the outer surfaces of the ports 120 and 130 are male threaded surfaces. The ports 120 and 130 and of their respective male threaded surfaces are sized and shaped to threadingly engage and disengage the female threaded surface of the distal end 111 of the OPG 100. As one alternative, the distal end 111 could be a male threaded surface and the threaded surfaces of the ports 120 and 130 could be female threaded surfaces.

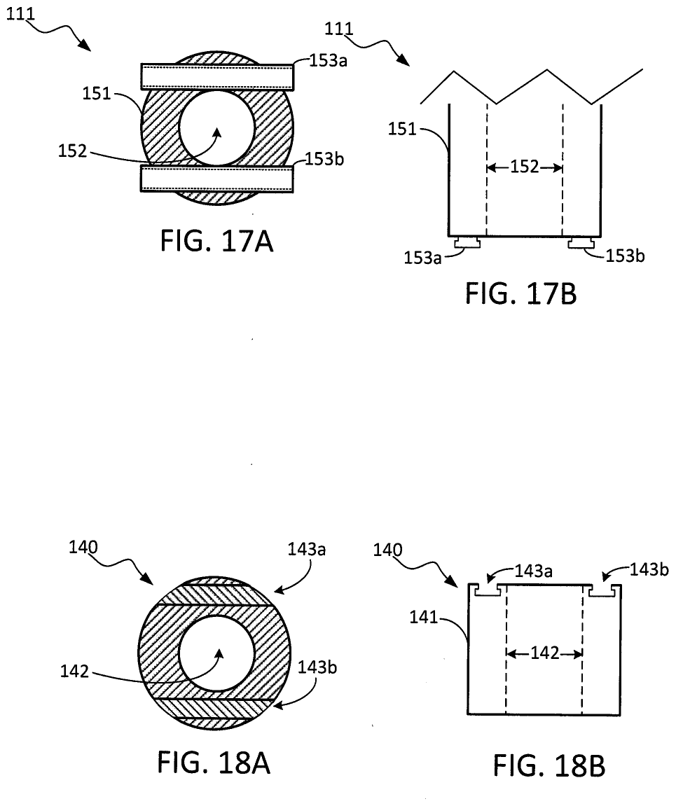

These threaded configurations represent one of many possible removably-attachable configurations for making the distal ends ports removably attachable to, and detachable from, the distal end 111 of the OPG 100, as will be understood by those skilled in the art in view of the discussion provided herein. Any other suitable attachment/detachment configurations can be used for this purpose, including, but not limited to, mechanical latching features, snap fit features, friction-fit features, sliding engagement features, permanent magnetic features, electromagnetic features, adhesives, etc. Any attachment/detachment configuration that is used for this purpose should be capable of remaining securely attached during the bronchoscopy procedure. Another possibility is discussed below with reference to FIGS. 17A-18B.

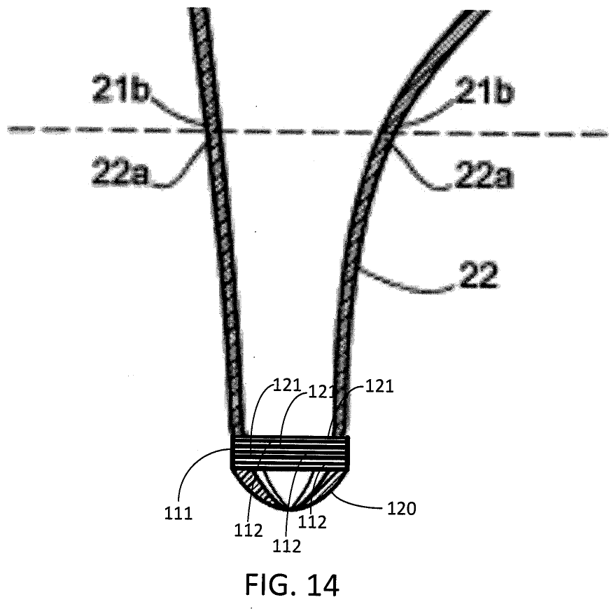

FIG. 14 is a side cross-sectional view of the second membrane portion 22 of the OPG 100 shown in FIG. 11 in accordance with a representative embodiment in which the distal end 111 of the OPG 100 is threadingly engaged with the removably-attachable distal end port 120 shown in FIG. 12 to enable the OPG 100 to be used for rigid bronchoscopy. The lower portion 120b of the port 120 can be identical to, and serve the same purpose as, the second opening 7 of the distal end 8 described above with reference to FIGS. 2 and 3.

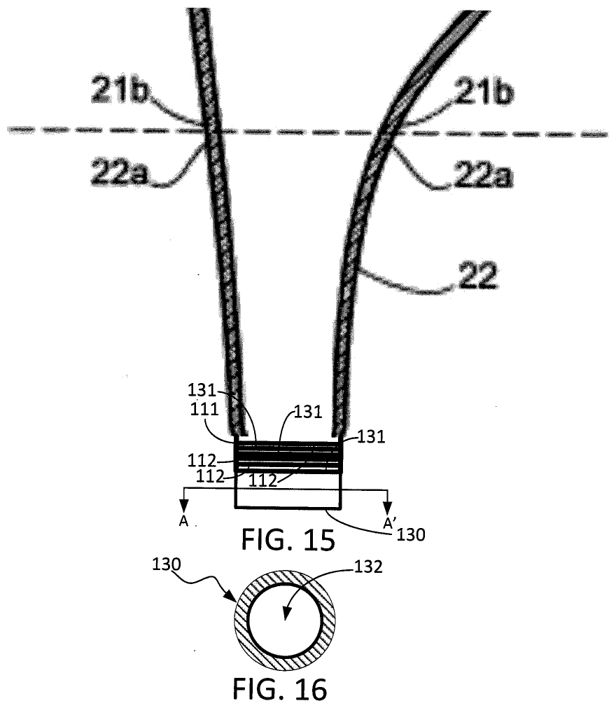

FIG. 15 is a side plan view of the second membrane portion 22 of the OPG 100 shown in FIG. 11 in accordance with a representative embodiment in which in which the distal end 111 of the OPG 100 is threadingly engaged with the removably-attachable distal end port 130 shown in FIG. 13 to enable the OPG 100 to be used for flexible bronchoscopy. FIG. 16 is a bottom end view of a cross-section of the removably-attachable distal end port 130 shown in FIG. 15 taken along line A-A′ of FIG. 15 in accordance with a representative embodiment. Because the flexible tube of the flexible bronchoscope will typically not be sufficiently stiff to press through the port 120 shown in FIG. 12, the port 130 has a tubular or cylindrical shape similar that of the tubular extension 101 shown in FIGS. 8 and 9 having an inner hollow bore 132 through which the flexible bronchoscope tube can pass. The diameter of the inner bore 132 is larger than the diameter or width of the flexible bronchoscope tube to allow the flexible bronchoscope tube to easily pass through the port 130.

FIGS. 17A and 17B show bottom end and side plan views, respectively, of the distal end 111 of the OPG 100 shown in FIG. 8 in which the distal end 111 has been modified to slidably engage a removably-attachable distal end port to enable the OPG 100 to be used for flexible bronchoscopy. FIGS. 18A and 18B show bottom end and side plan views, respectively, of a removably-attachable distal end port 140 in accordance with a representative embodiment adapted to slidably engage the distal end 111 of the OPG 100 shown in FIGS. 17A and 17B to enable the OPG to be used for flexible bronchoscopy.

As indicated above, the threaded configurations discussed above represent one of many possible removably-attachable configurations for making the distal ends ports removably attachable to, and detachable from, the distal end 111 of the OPG 100. FIGS. 17A-18B Another possibility is discussed below with reference to FIGS. 17A-18B. FIGS. 17A-18B represent another example of a suitable attachment/detachment configuration. Although the distal end port is not required to be removably attachable, removable attachability of the distal end port allows the same OPG 100 to be used for flexible and rigid bronchoscopy, and thereby obviating the need to design and manufacture different OPGs for flexible and rigid bronchoscopy, which reduces costs.

With reference to FIGS. 17A and 17B, the distal end 111 has a substantially tubular section 151 having an inner hollow bore 152 and first and second rails 153a and 153b, respectively, disposed on the bottom surface thereof. With reference to FIGS. 18A and 18B, the port 140 has a substantially tubular section 141 having an inner hollow bore 142 and first and second tracks 143a and 143b, respectively, disposed on the bottom surface thereof. The inner hollow bore 142 is large enough in diameter or wide enough for a flexible bronchoscope tube to pass through it.

The tracks 143a and 143b are complementary in shape and size to the rails 153a and 153b, respectively, to allow the tracks 153a and 153b to slidingly engage the rails 143a and 143b, respectively, thereby removably attaching the distal end port 140 to the distal end 111 of the OPG 100. It should be noted that the positions of the tracks 153a and 153b and the rails 143a and 143b can be swapped such that the rails 143a and 143b are disposed on the bottom side of the section 151 of the distal end 111 and the tracks 153a and 153b are disposed on the top side of the port 140. As indicated above, many other attachable/detachable configurations are also possible, a few non-limiting examples of which have been mentioned above.

When the OPG 100 is being used for rigid bronchoscopy and anesthesia recovery, a tube of the breathing circuit that is connected to the ventilator machine is connected to the tubular extension 101 after the rigid bronchoscope tube has been removed from the OPG 100 to continue supplying air to the patient's airway. When the OPG 100 is being used for flexible bronchoscopy and anesthesia recovery, the tube of the breathing circuit that is connected to the ventilator machine is connected to the tubular extension 101 prior to performing the bronchoscopy procedure. After the breathing circuit is connected to the tubular extension, the tube of the flexible bronchoscope can be inserted via a port of the breathing circuit through the tubular extension 101 into the patient. Air is supplied to the patient through the tubular extension 101 during the bronchoscopy and post procedure during anesthesia recovery. After the flexible bronchoscope procedure has been performed, the tube of the flexible bronchoscope can be retracted through the port of the breathing circuit and the port can be capped to prevent air leakage during recovery.

It should be noted that the inventive principles and concepts have been described with reference to representative embodiments, but that the inventive principles and concepts are not limited to the representative embodiments described herein. Although the inventive principles and concepts have been illustrated and described in detail in the drawings and in the foregoing description, such illustration and description are to be considered illustrative or exemplary and not restrictive; the invention is not limited to the disclosed embodiments. Other variations to the disclosed embodiments can be understood and effected by those skilled in the art, from a study of the drawings, the disclosure, and the appended claims.

What is claimed is:

1. An oropharyngeal glove (OPG) configured for use with a bronchoscope that is passed through the OPG into a patient's trachea during a bronchoscopy procedure, the OPG comprising:
a flexible body having a proximal end, a distal end, and first and second openings at the proximal and distal ends, respectively, the flexible body being configured to conform to a patient's mouth and throat when the OPG is installed in a patient's mouth and throat such that outer walls of the flexible body are in contact with features of the patient's mouth and throat, the flexible body being configured to form a seal with the features of the patient's mouth and throat with which the flexible body is in contact and to seal an upper esophageal aperture of the patient's throat, the seal being configured to prevent or reduce air leakage from the patient's trachea and to prevent or reduce aspiration of gastric contents into the patient's trachea while the patient is under anesthesia, the OPG protecting the patient's mouth and throat from being damaged by a tube of the bronchoscope during a bronchoscopy procedure, and wherein the OPG is configured for the tube of the bronchoscope to pass through the first opening, through the flexible body and through the second opening during a bronchoscopy procedure;
a tubular extension having a proximal end, a distal end, and a tubular section that extends from the proximal end of the tubular extension to the distal end of the tubular extension, the tubular section of the tubular extension having a hollow inner bore that extends from the proximal end of the tubular extension to the distal end of the tubular extension, the distal end of the tubular extension interfacing with the proximal end of the flexible body such that the hollow inner bore is substantially aligned with the first opening of the flexible body, the hollow inner bore having an inner diameter corresponding to an inner diameter of the tubular extension, the inner diameter being sufficiently large to allow the tube of the bronchoscope to pass through the hollow inner bore and into the first opening during a bronchoscopy procedure; and
wherein the tubular extension is configured to couple with a ventilator machine via one or more tubes of a breathing circuit and to act as an airway device to deliver air produced by the ventilator machine to the patient after completion of the bronchoscopy procedure.

2. The OPG of claim 1, further comprising upper and lower teeth guards disposed at the proximal end of the flexible body on opposite sides of the first opening and configured to protect upper and lower teeth, respectively, of the patient.

3. The OPG of claim 1, wherein the tubular section of the tubular extension has an outer diameter that is selected to interconnect with a connecting tube of said one or more tubes of the breathing circuit such that an airtight or substantially airtight seal exists between an outer surface of the tubular section and an inner surface of the connecting tube.

4. The OPG of claim 1, wherein the inner diameter of the tubular section is selected to interconnect with a connecting tube of said one or more tubes of the breathing circuit such that an airtight or substantially airtight seal exists between an inner surface of the tubular section and an outer surface of the connecting tube.

5. The OPG of claim 1, wherein the distal end of the flexible body is configured to be removably attached to a removably-attachable distal end port that is configured to enable at least one of a tube of a rigid bronchoscope and a flexible bronchoscope to pass therethrough.

6. The OPG of claim 5, wherein the distal end of the flexible body is configured to be removably attached to a removably-attachable distal end port that is configured to enable a tube of a rigid bronchoscope to pass therethrough and to create an airtight or substantially airtight seal between an outer surface of the tube of the rigid bronchoscope and the removably-attachable distal end port.

7. The OPG of claim 6, wherein the configuration of the distal end of the flexible body is a female threaded configuration and wherein the configuration of the removably-attachable distal end port is a male threaded configuration sized to mate with the female threaded configuration.

8. The OPG of claim 6, wherein the configuration of the distal end of the flexible body is a male threaded configuration and wherein the configuration of the removably-attachable distal end port is a female threaded configuration sized to mate with the male threaded configuration.

9. The OPG of claim 6, wherein the configuration of the distal end of the flexible body is a snap-fit configuration and wherein the configuration of the removably-attachable distal end port is a snap-fit configuration sized and shaped to mate with the snap-fit configuration of the distal end of the flexible body.

10. The OPG of claim 6, wherein the configuration of the distal end of the flexible body is a sliding-engagement configuration and wherein the configuration of the removably-attachable distal end port is a sliding-engagement configuration sized and shaped to mate with the sliding-engagement configuration of the distal end of the flexible body.

11. The OPG of claim 5, wherein the distal end of the flexible body is configured to be removably attached to a removably-attachable distal end port that is configured to enable a tube of a flexible bronchoscope to pass therethrough without interference.

12. The OPG of claim 11, wherein the configuration of the distal end of the flexible body is a female threaded configuration and wherein the configuration of the removably-attachable distal end port is a male threaded configuration sized to mate with the female threaded configuration.

13. The OPG of claim 11, wherein the configuration of the distal end of the flexible body is a male threaded configuration and wherein the configuration of the removably-attachable distal end port is a female threaded configuration sized to mate with the male threaded configuration.

14. The OPG of claim 11, wherein the configuration of the distal end of the flexible body is a snap-fit configuration and wherein the configuration of the removably-attachable distal end port is a snap-fit configuration sized and shaped to mate with the snap-fit configuration of the distal end of the flexible body.

15. The OPG of claim 11, wherein the configuration of the distal end of the flexible body is a sliding-engagement configuration and wherein the configuration of the removably-attachable distal end port is a sliding-engagement configuration sized and shaped to mate with the sliding-engagement configuration of the distal end of the flexible body.

16. The OPG of claim 2, wherein the flexible body comprises a first membrane portion and a second membrane portion, the first membrane portion having a first end that joins the upper and lower teeth guards and having a second end that joins a first end of the second membrane portion, the second membrane portion having a second end that extends to the second opening disposed at the distal end of the OPG, wherein the OPG is configured such that when the OPG is installed in the patient's mouth and throat, outer walls of the first membrane portion are in contact with the patient's tongue, with inner walls of cheeks of the patient and with a roof of the patient's mouth, wherein when the OPG is configured such that when the OPG is installed in the patient's mouth and throat, outer walls of the second membrane portion are in contact with the patient's throat.

17. The OPG of claim 16, wherein the first membrane portion has a circumference that is greater than a circumference of the second membrane portion.

18. The OPG of claim 16, wherein the OPG is configured such that when the OPG is installed in a patient's mouth, the distal end of the flexible body is positioned in between vocal cords of the patient and an epiglottis of the patient.

19. A method for performing a rigid or flexible bronchoscopy procedure and for performing anaesthesia recovery after the bronchoscopy procedure, the method comprising:
installing an oropharyngeal glove (OPG) in a patient's mouth and throat, the OPG comprising:
a flexible body having a proximal end and a distal end, the proximal end having a first opening therein, the distal end having a second opening therein; and
a tubular extension having a proximal end, a distal end, and a tubular section that extends from the proximal end of the tubular extension to the distal end of the tubular extension, the tubular section having a hollow inner bore that extends from the proximal end of the tubular extension to the distal end of the tubular extension, the distal end of the tubular extension interfacing with the proximal end of the flexible body such that the hollow inner bore is substantially aligned with the first opening of the flexible body, the hollow inner bore having an inner diameter corresponding to an inner diameter of the tubular extension, the inner diameter being sufficiently large to allow a tube of the bronchoscope to pass through the hollow inner bore and into the first opening during a bronchoscopy procedure;
inserting a tube of a bronchoscope through the hollow inner bore, through the first and second openings and into a trachea of the patient;
manipulating the tube of the bronchoscope to perform a bronchoscopy procedure, wherein the OPG protects the mouth and throat of the patient from being injured by the tube of the bronchoscope;
removing the tube of the bronchoscope from the OPG; and
with a ventilator machine coupled via one or more tubes of a breathing circuit to the proximal end of the tubular extension, using the tubular extension as an airway device to deliver air produced by the ventilator machine to the patient to perform anaesthesia recovery.

20. The method of claim 19, wherein the flexible body is configured to conform to a patient's mouth and throat when the OPG is installed in a patient's mouth and throat such that outer walls of the flexible body are in contact with features of the patient's mouth and throat, the flexible body being configured to form a seal with the features of the patient's mouth and throat with which the flexible body is in contact and to seal an upper esophageal aperture of the patient's throat, the seal being configured to prevent or reduce air leakage from the patient's trachea and to prevent or reduce aspiration of gastric contents into the patient's trachea while the patient is under anesthesia, the OPG protecting the patient's mouth and throat from being damaged by a tube of the bronchoscope during a bronchoscopy procedure, and wherein the OPG is configured for the tube of the bronchoscope to pass through the first opening, through the flexible body and through the second opening during a bronchoscopy procedure.
